(19) United States
(12) Patent Application Publication
(10) Pub. No.: US 2017/0326284 A1
(43) Pub. Date: Nov. 16, 2017

(54) Method for Controlling a Blood Treatment Device While Considering Patient Feedback in the Treatment of Blood and Blood Treatment Device

(71) Applicant: Fresenius Medical Care Deutschland GmbH, Bad Homburg (DE)

(72) Inventors: Erik Dülsner, Florstadt (DE); Alexander Schrörs, Frankfurt am Main (DE); Wolfgang Wehmeyer, Tübingen (DE)

(21) Appl. No.: 15/512,214

(22) PCT Filed: Sep. 17, 2015
(86) PCT No.: PCT/EP2015/071325

§ 371 Date: Jul. 27, 2017

(30) Foreign Application Priority Data
Sep. 18, 2014 (DE) 10 2014 113 462.1

(51) Int. Cl. A61M 1/16; A61M 1/34; G06F 19/00

(52) U.S. Cl. CPC A61M 1/1605; A61M 1/3406; A61M 1/341; A61M 1/3413; A61M 1/1603; A61M 2205/3368; A61M 2205/505; G06F 19/3406; A61M 2205/3334

(57) ABSTRACT

A control and/or regulation device, which is connected via signal link to a first input device, which in particular is operable by a doctor, and to a second input device, which in particular is operable by a patient, wherein the control or regulation device is programmed to control and/or regulate in signal link with a blood treatment

### CROSS-REFERENCE TO RELATED APPLICATIONS

This application is the national stage entry of International Patent Application No. PCT/EP2015/071325, filed on Sep. 17, 2015, which claims priority to German Patent Application No. 10 2014 113 462.1, filed on Sep. 18, 2014, the entire contents of which are hereby incorporated by reference.

### TECHNICAL FIELD

The present disclosure concerns a control and/or regulation device and a method as well as a blood treatment device.

### BACKGROUND

A blood treatment, for example in the form of dialysis, is often a procedure that represents a burden for the patient. The patient feels a burden because of—amongst other reasons—physical symptoms such as fatigue, headaches, nausea and further symptoms. All those symptoms can be exacerbated or can alternate during treatment. The blood treatment as such can—in addition—be burdensome to the patient, because of limitations, which the patient feels because of the necessity of treatment, the time he needs to set aside for his treatments and the feeling of powerlessness concerning his underlying disease, which necessitates the treatment, inter alia. The occurrence of some or all of the symptoms previously mentioned and their severity can be prevented or ameliorated by smart control or regulation of the blood treatment device used for the treatment.

### SUMMARY

Aspects of this disclosure relate to a further control and/or regulation device (in the following also, in short: control device) for a blood treatment device, as well as a method and a blood treatment device with such a control device.

The control and/or regulation device according to certain embodiments (in short control device) is connected to a first input device by signal link, in particular an input device which can be operated by a doctor, or is prepared for such a connection. The control device is furthermore connected to a second input device by signal link, in particular an input device which can be operated by a patient, or is prepared for such a connection. The control device according to some embodiments is programmed to be in signal link with a blood treatment device and/or to cause the blood treatment device to carry out a blood treatment. To this end, the blood treatment device is connected to a blood filter via an extracorporeal blood circuit.

The control device according to some embodiments is further programmed to control and/or regulate (in the following simply described as “control”) the patient's blood treatment or the blood treatment device by carrying out at least the steps mentioned in the following.

In this way, the blood treatment device for the patient's blood treatment is controlled with a first setting, described in here as original setting, for at least one treatment parameter. The first setting is that input or instruction, which is set up, entered or pre-set via the first input device, directed to the blood treatment device.

The control device is programmed to check once, several times, in regular intervals, particularly in certain predetermined time intervals, whether an input occurred via the second input device.

If it is detected that such an input has occurred, the control device evaluates the input on account of its programming; if no input has occurred there is no evaluation. Alternatively or in addition to this evaluation, a second setting is specified by the control device, described herein as target setting, for the at least one treatment parameter. The specification is based on—or is carried out depending on—the input made via the second input device (if such an input was made, otherwise this step is not required); this input is taken into account for the specification, for example in a computational manner or by using a look-up table.

If a target setting was specified via the control device, the control device controls the blood treatment device —preferably from the moment of the specification, alternatively delayed—in such way that the further blood treatment of the patient occurs with the target setting and thus with the second setting.

The specification of the target setting can be understood as an adjusting, changing or overwriting of the original setting.

The method according to some embodiments for controlling a blood treatment device comprises a provision of a control and/or regulation device, which is connected via signal link to a first input device, which in particular is operable by a doctor, and to a second input device, which in particular is operable by a patient.

The control or regulation device is programmed to control and/or regulate in signal link with a blood treatment device, which, in turn, is connected to a blood filter, a blood treatment of the patient while executing the following steps:
controlling and/or regulating of the blood treatment device for the blood treatment of the patient with a first setting, described as original setting, set by using the first input device for at least one treatment parameter;
checking, whether an input has occurred using the second input device;
evaluating the input entered using the second input device and/or specifying of a second setting, described as target setting, for the at least one treatment parameter based on the input entered using the second input device; and
controlling or regulating the blood treatment device for the blood treatment of the patient with the second setting and/or controlling or regulating of the blood treatment device while adjusting or changing of the original setting according to the input or the target setting.

The blood treatment device according to some embodiments has an extracorporeal blood circuit and/or a blood cassette or is connected to such. The blood treatment device further has at least one control and/or regulation device according to some embodiments.

Embodiments can present some or all of the following features in any combination as long as this is not technically impossible as seen by the person skilled in the art. Features mentioned herein in conjunction with the device can be combined with the method and vice versa.

In all of the embodiments mentioned above or below, the use of the expressions “may be” and “may have” etc. is synonymous to the expressions “is preferably” and “has preferably”, respectively and is intended to illustrate embodiments.

Wherever numbers are mentioned herein, the skilled person will understand these as the specification of a lower limit. As long as this does not lead to a contradiction recognisable by the person skilled in the art, the skilled person will thus read “one” invariably as “at least one”. This understanding is comprised by the present invention just as the understanding that a number, such as “one”, can alternatively be read as “exactly one”, wherever this is technically possible as recognisable by the person skilled in the art. Both is comprised by the present invention and is thus valid for all numbers comprised herein.

Whenever the expressions “suitable”, “provided”, “interpreted”, “configured” and/or “programmed” are mentioned herein, the skilled person will see this as a specific embodiment of the device in question. The aforementioned terms can be used interchangeably herein.

Even though embodiments of the invention are described in connection with dialysis treatment in the following, the invention is not limited to this.

In some exemplary embodiments, the control device is programmed to permit an input via the second input device, the specification of the target setting and/or the adjusting or changing of the original setting of the at least one treatment parameter only within a specified frame or area—for example set by the factory or by staff and/or via the first input device—or within previously specified limits.

In certain, exemplary embodiments, the control device is programmed to be able to enter a frame or range or limits, within which the input via the second input device, the specification and/or adjusting of the at least one treatment parameter is possible or made possible.

In certain, exemplary embodiments, the treatment parameter is chosen from the group consisting of: flow of the dialysis liquid, blood flow through the blood filter; inflow or inflow rate of an infusion solution, provision or concentration of osmotically active substances in the dialysis liquid or the substitution fluid, concentration of bicarbonate, sodium, potassium, magnesium and/or calcium, each in dialysis liquid or substitution fluid, filtration rate, ultrafiltration rate, duration of the blood treatment, temperature of the blood reinfused into the patient and/or temperature of the dialysis liquid. The temperature is for example that of a source of dialysis liquid or that of the dialysis liquid at its entry into the blood filter or into the extracorporeal blood circulation.

The blood treatment device can possess all devices required for carrying out the control and/or regulation, such as blood pump, ultrafiltration pump or the like. The control device can be in signal link with these devices.

Included in the treatment parameters, which can be influenced by the patient, is—directly or indirectly—the temperature, with which the blood is returned to the patient. This temperature is a factor, which influences the physical comfort of the patient. On the other hand, the blood temperature has a therapeutic effect, which the patient can influence by his inputs.

The dialysis patient's doctor, for example, tends to set the temperature, which the dialysis liquid should have, to set it in a way that leads to a slight cooling or hypothermia of the patient. This is done—on the one hand—in the intention to dissipate heat created by the ultrafiltration in the body core. On the other hand, this is done to produce a slight vasoconstriction of the peripheral vessels brought on by the cold in order to support the blood pressure during dialysis. If the temperature is too low, the patient will complain about being too cold. Furthermore, if this is the case, a certain limitation of the dialysis quality is to be expected as due to the vasoconstriction a pool in the body was not detoxified well. If the temperature is too high, a dangerous decrease of blood pressure can occur during dialysis due to vasodilation.

According to some embodiments, the doctor sets a temperature range at first and individually for each patient, which avoids the previously stated effects. This temperature range is smaller than the temperature range, which can be set in the blood treatment device. The dialysis is then started with a temperature value for the dialysis liquid, which is for example the mean of those limits.

The patient can modify the temperature of the dialysis liquid in any way via the second input device during the treatment and preferably within the limits set by the doctor.

In this, it is not required in certain, exemplary embodiments that the patient knows the absolute temperature when he modifies the temperature. Thus, this temperature does not need to be determined or communicated to the patient. It may be sufficient if the parameter adjustable via the second input device is expressed in relative terms, such as “warmer” or “colder”.

In some exemplary embodiments, a target setting is only used temporarily as a basis for the control. By stepwise or continuous adjustment, the control will be carried out on the basis of the original setting again, after an input by the patient using the second input device. The duration, after which this should be the case, can be adjustable. The transition from target setting to the original setting can be carried out step by step according to a previously set pattern, it can occur stepwise, etc.

In some exemplary embodiments, the range or area or the limits, within which the input via the second input device, the specification and/or the adjusting of the at least one treatment parameter is made possible, for example, in a supplementary fashion or alternatively, depending on the result of blood pressure measurements carried out during the blood treatment, is further modified automatically, preferably limited further. To this end, the result of the blood pressure measurement can be compared with the blood pressure limits previously determined for said patient to be admissible. If the limits are exceeded, the parameter range available for input or target setting, for example the temperature range of the dialysis liquid, will be adjusted.

In certain exemplary embodiments, the second input device is based on a mechanical and/or electric solution. It can, for example, be a linear or rotary potentiometer. It can be a switch or toggle switch. It can be an acoustic input device (speech input). It can be operable using a remote control and/or gesture control. It can possess a screen and/or can be operable via touch (for example as a touch panel).

In some exemplary embodiments, the second input device is designed or configured to at least or exclusively allow an input, which is qualitative, which causes a qualitative adjustment or codes for it. The control device is programmed to transform the qualitative input entered via the second input device according to predetermined routines or rules into quantitative target settings.

In certain exemplary embodiments, the second input device allows no quantitative setting. In other embodiments, the second input device exclusively allows quantitative inputs, that is inputs which cause a quantitative adjustment or code for such.

Quantitative inputs can be marked as “warmer” or “colder”, see above. They can also be marked “more gentle”, “more efficient”, “slower” or “faster” or the like.

According to stored rules or routines, each and every quantitative input can lead to foreseeable changes of the control regarding one or several adjustable treatment parameters. In this way, the operation of a “more gentle” switch can lead to a lowering of the pump power of the ultrafiltration power (and optionally of further treatment parameters), as predetermined, while the operation of a “colder” switch can lead to a lowering of the temperature of the dialysis liquid.

The second input device offers the patient in certain exemplary embodiments more than only one qualitative input possibility, optionally with opposite effect, where applicable. Rather, the input device can be designed to allow a “more gently/more efficient” input in addition to a “warmer/colder” input, etc.

In some exemplary embodiments, the control device is designed in such a way that, for the input via the second input device, it is possible to choose between at least or exclusively a first and a second input option, which preferably each causes an adjusting or changing of the original setting of the treatment parameter in opposite direction, for example as compared to the present value or original setting of the treatment parameter or blood treatment device.

In some exemplary embodiments, the adjustment or the changing of the settings causes a predetermined change of the treatment parameter, particularly by a fixed value or number.

In certain exemplary embodiments, the adjustment or changing of the original settings is a variable adjustment, for example via controller/regulator, touch screen, tablet input or slider, wherein preferably a continuous adjusting, preferably an adjusting with at least several steps, is possible (for example by providing at least 3, 4 or more selectable steps for the adjustment).

In some exemplary embodiments, the control device is programmed to effect the adjustment or the changing of the original setting of the treatment parameter and/or the specification of the target setting depending on the time passed since the beginning of the blood treatment.

For example, it can be provided that the operation by the patient of a “more effective” switch as a second input device leads to a more gentle increase, for example of the pumping rates, at an earlier point in time during the treatment session than the same input would lead to at a later point in time. This embodiment is based on, for example, the concept that the more gentle increase of the pumping rates can have the same result at an earlier point in time as a larger increase of the pumping rates at a later point in time, at which point, if the end time of the treatment is fixed, a comparatively shorter time remains to achieve a higher efficiency.

In some exemplary embodiments, the control device is programmed to compensate an adjustment or changing of the original setting of the treatment parameter, which adjustment or changing is carried out via the second input device at a first point in time or during a first time period during the blood treatment, at a second point in time or during a second time period during the blood treatment. To this end, the control device is programmed to control or regulate the blood treatment at the second point in time or second time period automatically with a third setting, described here as a correction setting. In this, the second point in time or the second time period are after the first time point or time period, respectively.

In some exemplary embodiments, the correction setting is defined such that an aim of the blood treatment, targeted via an original setting, is achieved despite the input by the patient, particularly during a certain time period of the blood treatment.

In certain exemplary embodiments, the correction setting is defined such that an aim of the blood treatment, targeted via an original setting, is achieved within an originally set treatment duration, set for example before or at the beginning of the blood treatment.

In certain exemplary embodiments, the targeted, preferably predetermined or pre-defined aim can be a hydration condition and/or a detoxification condition, which, again, can be predetermined or pre-defined.

As is well-known, the hydration condition of the patient shows the water burden of the patient, which, in the case of overhydration, was not be discharged via the kidneys, and which, in the case of dehydration, the body is deficient of, as compared to a physiologic hydration. Hyperhydration (or overhydration) and dehydration (or water deficiency) are both signs of an abnormal fluid volume regulation, as this is explained, for example, in Guyton A C, Hall J E, Physiology, Elsevier & Saunders, lied, (2006), page 301. Methods and a device for measuring a hydration condition are known for example from WO 2006/002685 A1. Therein, an overhydration is calculated as a*ECW+b*ICW+c*body weight, wherein ECW is extracellular water, ICW is intracellular water, a, b, and c are variables or constants.

The detoxification condition is that condition, in which a physiologic concentration or a concentration, which was set before the start of the blood treatment as a treatment target, of one or several substances in the blood, such as urea, potassium or sodium, is achieved.

In certain exemplary embodiments, the correction setting is specified such that an effect of the deviation from the original setting, which was caused by the controlling according to the target setting for a certain time period, is at least partly compensated, cancelled or the effect of the deviation is counteracted.

In some exemplary embodiments, the control device is programmed to, after the controlling or regulating of the blood treatment device according to the second setting or the target setting, automatically and at least partially return from the target setting to the original setting. This can, for example, occur progressively. It can occur in its entirety or only partially.

In certain exemplary embodiments, the control device is programmed, after controlling or regulating of the blood treatment device according to the second setting has been initiated, to postpone the end time, which was predetermined or calculated by the control or regulating device, depending on the target setting. Alternatively, the treatment duration, which was predetermined or calculated by the control or regulation device, can be extended automatically depending on the target setting.

In some exemplary embodiments, the control device is programmed in such a way that an input made via the second input device and/or the specification of the target setting can only occur, if the input is made during at least one predetermined time period of the blood treatment. In other time periods than the predetermined one(s), such an input is not possible, will not be evaluated or does not lead to the specification of a target setting.

In certain exemplary embodiments, the control device is programmed in such a way that an input made via the second input device can be made by speech and/or gesture.

In some exemplary embodiments, the control device possesses an output device for outputting and/or displaying of an optical or acoustic feedback or presentation of this input or its effects (for example changing of the Kt/V or of the ultrafiltration rate), respectively, after their calculation for the information of the patient or the nursing staff. Further aspects of the input can be communicated, such as an extension of the treatment duration necessitated by the input or other effects of the input on the treatment. In this way, the patient can prepare himself early on regarding the further treatment course, which could, for example, have been changed as compared to the initial planning.

In certain exemplary embodiments, the control device possesses at least one first input device and a second input device or is connected to both, which are each configured for setting or changing of the settings of at least one, particularly the exact same, treatment parameter.

In certain exemplary embodiments, the blood treatment device is a haemodialysis device, haemofiltration device or haemodiafiltration device.

In some embodiments, the control device is designed as a regulation device.

The blood treatment device possesses in certain embodiments at least one control device.

Some or all of the embodiments can possess one, several or all of the advantages stated above and/or below.

All of the advantages achievable by the control device can be achieved in certain embodiments by the blood treatment device in an undiminished way.

An advantage achievable by some embodiments is that the patient can be involved more deeply in the control of his blood treatment, for example a dialysis treatment. In this way, he is given the opportunity to take over more responsibility for himself and to gain a more positive attitude to the treatment situation, which can often be burdensome.

By wielding influence, the patient can remove himself from the situation, in which he often feels helpless and can experience himself as the person in charge, by which he becomes a more competent partner in the dialogue with the doctor and the nursing staff.

In addition to this, the patient can free the staff from some tasks by using the second input device of the devices, as he can carry out, within certain limits, such adjustments of his treatment, which he feels are necessary. The devices and methods described herein advantageously enable the easing of burden on the staff, an increased physical comfort of the patient by the adjustment of the blood treatment settings, which he can carry out by himself more precisely and more frequently, while there is at the same time a strengthening of the individual responsibility of the patient, which is, in general, well-received.

As is well known, each person feels differently each day, depending on their condition. The settings of the blood treatment parameters at the blood treatment device, which are always the same, do not take this into account fully. Advantageously, patients can now adjust the blood treatment parameters such that they can be treated according to their daily condition.

One advantage is to offer the blood treatment patient, for example the dialysis patient, a possibility to influence his treatment. The possibility of influencing can be limited to a range within therapeutic limits. In some embodiments, the therapeutic target previously aimed at (in the case of dialysis for example the dialysis dose or the dehydration) will possibly not be reached.

In certain cases, the patient can influence via the second input device that the blood treatment, for example the dialysis treatment, in such advantageous way that the therapeutic targets set by the doctor by the first input device are exceeded. This is the case, for example, if the patient's circulation is especially robust or if the patient reacts in a “robust” way to highly effective treatments. This “robustness” is detected by the patient himself according to his current well-being, however, the patient remains under permanent supervision by the surveillance of physiologic parameters, such as blood pressure.

The patient can further influence the stress exerted upon him by the blood treatment directly and timely, if applicable long before a breakdown of the circulation could take place with its severe consequences. Thereby, complications are avoided, for example an emergency bolus for blood pressure increase (thereby, disadvantageously, the target weight is typically not reached with dialysis patients) or the creation of stenosis as a result of a fall in blood pressure (shortened lifespan of the vascular access).

When evaluating the point in time, at which the patient intervenes by operating the second input device, the most promising counteraction for decreasing the burden by the treatment can be induced automatically. The automation can support the training of the nursing staff for safely carrying out of the first aid measure displayed. It can further lighten the staff's workload.

Advantageously, the entry of settings for the blood treatment at the blood treatment device can take place in the language of the symptoms of the patient. In such embodiments, the input of or change of settings of technical parameters is not required. The knowledge of the translation of symptoms into technical parameters does not need to be trained anymore, but is provided advantageously by the control device.

During dialysis, the temperature of the dialysis liquid is set such that, if possible, no heat is transferred to the patient nor is heat dissipated away from the patient by any significant measure. To this end, the temperature of the dialysis liquid for the dialysis is predetermined by the doctor.

Depending on the environmental conditions (for example on a hot summer's day or a cold winter's day), a setting made can lead to the patient being cold or sweating. An adjustment of the temperature setting can thus be necessary during dialysis. Up to now, such a setting could, technically and organisationally, only be made by the nurse.

The patient can adjust the temperature at any time via the second input device according to his physical comfort, without being in conflict with the intention of the doctor's prescription.

### BRIEF DESCRIPTION OF THE FIGURES

In the following, embodiments are described purely exemplarily using the enclosed figures. In these figures, the same reference numbers refer to the same components.

FIG. 1 is a simplified representation of a blood treatment device with an extracorporeal blood circuit in a first embodiment.

FIG. 2 is a simplified representation of an exemplary treatment course, being carried out with the blood treatment device in a first embodiment.

FIG. 3 is a simplified representation of an exemplary treatment course, being carried out with the blood treatment device in a second embodiment.

FIG. 4 is a simplified representation of an exemplary treatment course, being carried out with the blood treatment device in a third embodiment.

### DETAILED DESCRIPTION

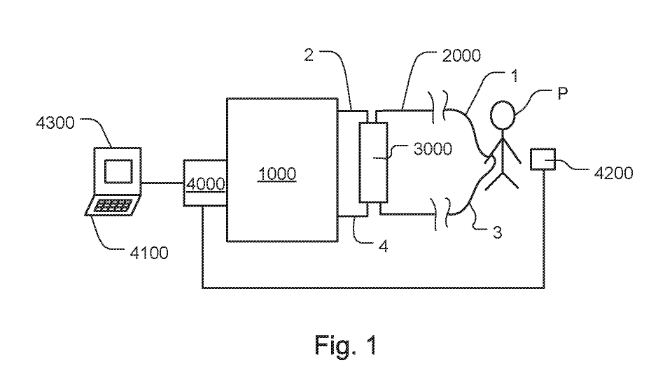

FIG. 1 shows in a simplified representation a blood treatment device 1000 with a source, which is not shown, for dialysis liquid and an extracorporeal blood circuit 2000.

The extracorporeal blood circuit 2000 possesses a blood treatment device 3000, here exemplarily a blood filter or dialyser, or is connected to such. An arterial line section 1 of the extracorporeal blood circuit 2000 carries blood from the vascular system of the patient P towards the blood filter 3000. A venous line section 3 of the extracorporeal blood circuit 2000 carries blood from blood filter 3000 towards the vascular system of the patient P.

The blood treatment device 1000 possesses a control and/or regulation device 4000 (in short: control device 4000) for controlling and/or regulating of the blood treatment being carried out using the blood treatment device 1000 or is connected to it by signal link.

The control device 4000 is connected by signal link with a first input device 4100 and with a second input device 4200. A display device 4300, on which settings regarding treatment parameters, the course of the blood treatment or entries made using the first and/or the second input device 4100, 4200 can be displayed, can be provided.

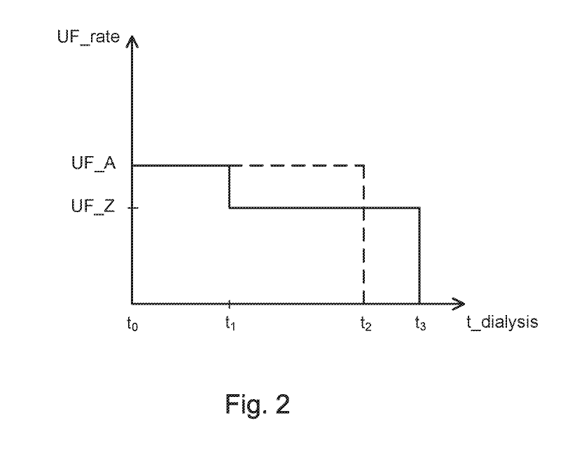

FIG. 2 shows in a simplified representation an exemplary treatment course being carried out using the blood treatment device 1000 in a first embodiment in a diagram, in which the solid line shows an actual ultrafiltration rate (UF_rate) of a dialysis treatment being carried out using a blood treatment device 1000 over a treatment time (t_dialyse).

The ultrafiltration treatment begins at a time t0 at the ultrafiltration rate UF_A (or UF original) entered by the doctor at the first input device 4100 as an original setting. At a time t1, the patient P, who desires a more gentle treatment than the one being carried out, submits a qualitative input (“gentle”, see the symbol “-”) using the second input device 4200, which leads to, after evaluation of the input and specification of the target setting, the ultrafiltration being carried out from that moment, deviating from the original setting, which is shown as a dashed line thereafter, at an ultrafiltration rate UF_Z (or UF target) as a target setting.

As a predetermined ultrafiltration performance or ultrafiltration amount is to be achieved, despite the change made or initiated by the patient at time t1 in the exemplary embodiment shown herein, the dialysis session of the patient extends beyond a time t2, previously considered to be the end of treatment, to a time t3, at which the predetermined ultrafiltration performance is achieved despite the input made by the patient.

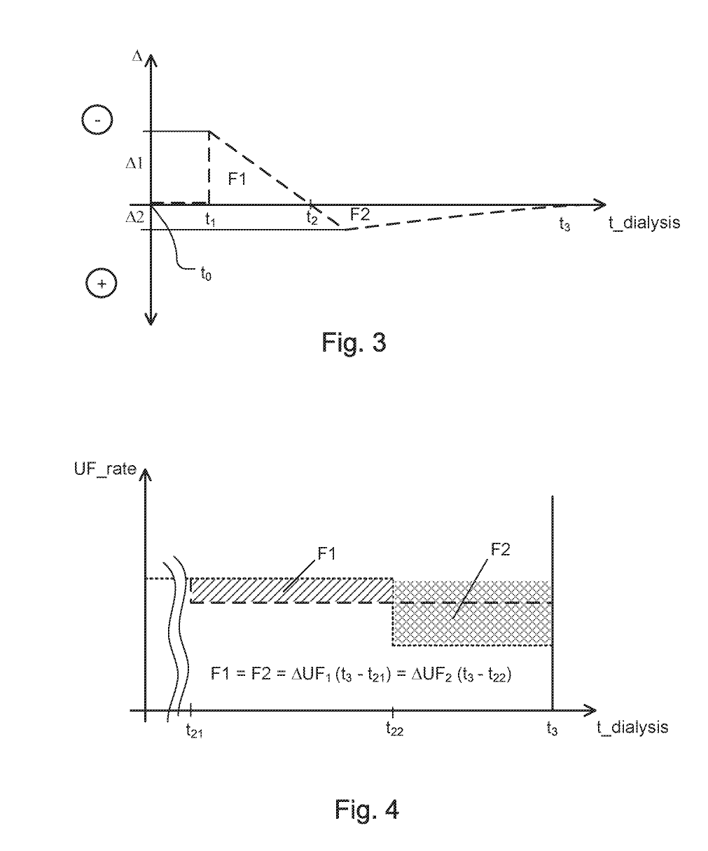

FIG. 3 shows in a simplified representation an exemplary treatment course being carried out using the blood treatment device in a second embodiment.

In a dialysis treatment, in principle, four processes running in parallel can be identified:

a) extraction of water
b) extraction of toxic substances
c1) correction of electrolytes (adjusting of sodium, potassium, magnesium and calcium)
c2) correction of the pH by adding of bicarbonate

The extraction processes a) and b) lead to imbalances in the body, which the patient can possibly feel and which, as a result, can lead to the known symptoms of feeling unwell.

However, imbalances during dialysis do not occur uniformly. Rather, a dialysis treatment can roughly be divided into essential phases 1 and 2: in phase 1, a strain is put on the patient by the extraction of osmotically active substances (“disequilibrium syndrome”). In phase 2, a strain is put on the patient by the extraction of water (with a possible severe hypotension).

In phase 1, the concentration gradient between blood and dialysis liquid in the blood filter 3000 is characteristic for the elimination speed of the osmotic substances. This is the highest at the start of the dialysis, at t0, and declines over time in a natural exponential function. As a result of the rapid extraction of water in phase 1, a concentration difference arises for osmotically active substances between the blood circulation and the surrounding tissue cells. This leads to, for example, the flow of water from the plasma into the cells of the brain and thus to an increase of pressure there, which can lead to headache. Similar mechanisms lead to cardiac arrhythmias due to the rapid decrease of potassium levels. In this phase, the patient can find relief, if he slows down the removal of osmotically active substances. To this end, he can make an input, described as “gentle”, using the second input device 4200, which takes the form of a regulator, slider, toggle switch or switch, for example. As a result, the blood treatment device 1000 reduces the diffusion of substances from the blood. To this end, one of the following measures—or any combination thereof—can be initiated:
reduction or stopping of the dialysis liquid flow
reduction of the blood flow through the blood filter
reduction of the flow rate of infusions applied
increase of the original concentration of the osmotically active substances in the dialysis liquid
reduction of the bicarbonate and/or of the sodium concentration in the dialysis liquid

FIG. 3 shows the result of a patient input in a representation of the deviation (A) over the duration of the dialysis (t_dialyse).

In the example of FIG. 3, the patient desired a “more gentle” treatment at time t1 and made a corresponding input. This input is “translated” via the control device 4000 into a target setting, with which the treatment is carried out from time t1 onwards.

The input can occur qualitatively either as a pulse or as a pulse with a quantitative intensity information. The input is translated simultaneously as pulse Δ1 for one or several of the parameters described previously. If the input represents a pulse then the value Δ1 is a fixed value, otherwise a specified translation (deposited in the software) was specified between the input of the patient P and the absolute value Δ1.

The fact that the strain by the osmotic changes on the body decreases degressively from the dialysis start at t0, leads to the following thoughts:

a) The fixed value for Δ1 as well as the translation of a variable input can be made to be dependent on time point t1, hence on the treatment time completed thus far. In certain embodiments, Δ1 can be decreased or amplified depending on the time, which has been completed since treatment start t0, by accessing the routines, algorithms or the like stored in the control device 4000. This way, it can be achieved that the felt impact of the intervention of the patient during the time period of phase 1 mostly corresponds to the desired benefit.
b) In the embodiment discussed here, the correcting intervention is retracted automatically until the end of phase 1 (t2, phase 1 lasts from t0 to t2; phase 2 starts from t2) by the control device 4000. Ideally, this occurs progressively, to counteract the degressive development of the concentration differences between blood and dialysis liquid. As a consequence, a linear concentration course of the osmotically active substances in the blood results, as shown in FIG. 3.

The intervention on the flow rates of the pumps used leads to a decrease of the cleaning performance (regarding the blood) in the current treatment session. The decrease in performance corresponds in FIG. 3 exemplarily to the triangular area designated F1.

As at the time t2, the concentration of toxic substances and electrolytes in the blood deviates from the desired value (shown in the time axis of FIG. 3) further than would be the case if the patient had not intervened, a certain catch-up-effect concerning the remaining time until the end of the dialysis at time t3 results automatically. However, this effect is not sufficient to achieve the same cleaning performance at the end of dialysis as would have been achieved without the interference occurred at t1. For this reason, optionally, a counter-correction of the parameters by Δ2 could occur in phase 2, to compensate this difference entirely or partially. The result of the counter-correction is to be understood in FIG. 3 as the triangular area designated F2.

FIG. 4 shows in a simplified representation an exemplary treatment course being carried out by the blood treatment device in a third embodiment.

In phase 1, see the remarks concerning FIG. 3, or generally in the initial phase of the dialysis treatment, the elimination of water causes usually few problems for the patient's body. The excessive water (the overhydration) is in the tissue as well as in the cells and in the vasculature. Due to this, the blood vessels are slightly expanded. The removal of water from the vasculature initially leads to a tension release, which the patient feels to be rather positive.

As more plasma water is removed from the vasculature, however, the blood pressure falls further. While water flows into the vessel lumina from the tissue surrounding the vessels, the body still tries to maintain blood pressure by constricting peripheral vessels and increased pulse frequency. This leads to an increased strain on the patient, which can be felt. As the case may be, the body is not able to compensate the mechanisms previously mentioned and the patient can become dangerously hypotensive.

Patient P can now take countermeasures, for example by reducing the ultrafiltration rate (UF_rate), that is the removal rate of fluid (for example indirectly via the “gentle/efficient”-regulator as the second input device 4200). In this way, the blood volume (the degree to which the vessels are filled) is increased and the blood pressure stabilised.

FIG. 4 shows the blood treatment in the phase described as phase 2 above. As in phase 1, the intervention of patient P, caused by the input into the second input device 4200, which occurs in FIG. 4 at time t21, can occur qualitatively as a pulse or quantitatively as intensity information.

The intervention regarding the ultrafiltration rate UF_rate can be interpreted by the software of the control device 4000 according to preferably at least three options: a), b) or c):

a) constant: that is, the ultrafiltration rate (UF_rate) remains limited until the end of the dialysis. As a consequence, there is a deviation from the prescribed dry weight after dialysis independent of the time point (could be t21, for example, or t22, for example, see FIG. 4), at which the patient intervened. FIG. 4 shows this option with each two different courses, shown once with a dotted line and once with a dashed line.
b) reducing: that is, the ultrafiltration rate UF_rate is returned by the end of the dialysis, that is until time point t3 increased to the regular value.
c) compensating: that is, the ultrafiltration rate UF_rate is returned and the remaining time of the dialysis is divided such that at the moment, less fluid is extracted, afterwards, however, more fluid is extracted, to achieve the dry weight prescribed by the doctor.

The choice of the options a) to c) as well as the maximum admissible height of the weight deviation at the end of the dialysis, and thus the admissible correction of the ultrafiltration rate UF_rate at the time of the intervention by the patient, is individually set for each patient by the doctor and stored in the software.

Regarding FIGS. 3 and 4, only the results of “more gentle” are described, which reduces the strain on the patient. Particularly concerning the ultrafiltration rate (UF_rate), “more efficient” is conceivable as well. Here, the ultrafiltration rate would be increased by a value and the dry weight at the end of the dialysis would be lower than the prescribed dry weight. The same mechanisms are valid, as described, in analogy. In this variant, the patient P understands “efficient” to be “allowed to drink more”.

As a technical result of this setting, the ultrafiltration rate (UF_rate) is corrected upwards already in phase 1, for example, if the patient P triggers “more efficient” at this time.

The above explanations concern the conceptual division of the treatment duration in two phases. It is also conceivable, however, to introduce more phases. For example, there could be a phase 1′ between phase 1 and phase 2, in which the operation of the second input device 4200, for example by setting “more gentle” or “more efficient”, has no influence, a target setting is thus not created. Such further phases, their start and end and the like can be specified in the setup of the blood treatment device 1000 and/or in the control device 4000.

If patient P operates the second input device 4200, for example within a certain time frame, several times, the control can convert each operation into a weight difference at the end of the dialysis, which can be restricted by a prescribed limit.

### LIST OF REFERENCE NUMBERS

1 arterial line section
2 dialysate line
3 venous line section
4 dialysis liquid line
1000 blood treatment device
2000 extracorporeal blood circuit
3000 blood treatment device, blood filter or dialyzer
4000 control or regulation device
4100 first input device
4200 second input device
4300 display device
P patient
t_dialyse treatment duration of a dialysis treatment
t0 start of treatment
t1 time point during the treatment; interference by the patient
t2 time point during the treatment
t3 end of treatment
UF_A ultrafiltration rate as initial setting
UF_rate ultrafiltration rate
UF_Z ultrafiltration rate as target setting

1-21. (canceled)

22. A control and/or regulation device, which is connected via signal link to a first input device, which is operable by a clinician, and to a second input device, which is operable by a patient, wherein the control and/or regulation device is programmed to control and/or regulate a blood treatment device that is connected to a blood filter by executing a method comprising:
controlling and/or regulating the blood treatment device for a blood treatment of a patient with a first setting set using the first input device for at least one treatment parameter;
checking whether an input has occurred using the second input device;
evaluating the input entered using the second input device and/or specifying a second setting for the at least one treatment parameter based on the input entered using the second input device; and
controlling or regulating the blood treatment device for the blood treatment of the patient with the second setting and/or controlling or regulating the blood treatment device while adjusting or changing the first setting according to the input or the second setting.

23. The control and/or regulation device according to claim 22, wherein the control and/or regulation device is programmed to allow the input using the second input device, the specifying of the target setting, and/or the adjusting or changing of the original setting of the at least one treatment parameter only within a previously specified frame or area or within previously specified limits.

24. The control and/or regulation device according to claim 22, wherein the treatment parameter is chosen from the group consisting of: flow of dialysis liquid; blood flow through the blood filter; inflow of an infusion solution; concentration of osmotically active substances in the dialysis liquid; concentration of bicarbonate, sodium, potassium, magnesium and/or calcium in the dialysis liquid; filtration rate; ultrafiltration rate; time period of the blood treatment; and temperature of the dialysis liquid.

25. The control and/or regulation device according to claim 22, wherein the second input device enables qualitative input to the control and/or regulation device, and wherein the control and/or regulation device is programmed to transform the qualitative input entered using the second input device according to predetermined routines or rules into the second setting.

26. The control and/or regulation device according to claim 22, wherein the control and/or regulation device is programmed such that when the second input device is used to enter a first input option or a second input option, the control and/or regulation device adjusts or changes the first setting of the treatment parameter.

27. The control and/or regulation device according to claim 22, wherein the control and/or regulation device is programmed such that the adjusting or changing of the first setting results in a predetermined change of the treatment parameter by a fixed value or number.

28. The control and/or regulation device according to claim 22, wherein the control and/or regulation device is programmed such that the adjusting or changing of the first setting comprises a variable and/or continuous adjustment.

29. The control and/or regulation device according to claim 22, wherein the control and/or regulation device is programmed to cause the adjusting or changing of the first setting of the treatment parameter and/or the specifying of the second setting depending on a time passed since a beginning of the blood treatment.

30. The control and/or regulation device according to claim 22, wherein the control and/or regulation device is programmed to control or regulate the adjusting or changing of the first setting of the treatment parameter by automatically adjusting or changing the first setting to a third setting, wherein the first setting occurred at a first time point or in a first time period during the blood treatment, the input was entered using the second input device at a second time point or in a second time period during the blood treatment, and the second time point or time period is after the first time point or time period, respectively.

31. The control and/or regulation device according to claim 30, wherein the third setting is determined such that a target of the blood treatment, aimed at by the first setting, is achieved.

32. The control and/or regulation device according to claim 31, wherein the target is a hydration state and/or a detoxification state of the patient.

33. The control and/or regulation device according to claim 30, wherein the third setting is determined such that an effect of a deviation from the first setting is compensated at least in part, is cancelled or is counteracted.

34. The control and/or regulation device according to claim 22, wherein the control and/or regulation device is programmed to return automatically at least in part from the second setting to the first setting after the controlling or regulating of the blood treatment device has taken place with the second setting.

35. The control and/or regulation device according to claim 22, wherein the control and/or regulation device is programmed to delay an end time point predetermined or calculated by the control or regulation device depending on the second setting or to extend a treatment duration predetermined or calculated by the control or regulation device depending on the second setting after the initiation of the controlling or regulating of the blood treatment device with the second setting.

36. The control and/or regulation device according to claim 22, wherein the control and/or regulation device is programmed such that the input made using the second input device and/or the specifying of the second setting only occurs if the input is made during a predetermined time period of the blood treatment.

37. The control and/or regulation device according to claim 22, wherein the control and/or regulation device is programmed such that the input made using the second input device can be made using speech and/or gestures.

38. A method of controlling a blood treatment device, the method comprising:
providing a control and/or regulation device, which is connected via signal link to a first input device, which is operable by a clinician, and to a second input device, which is operable by a patient, wherein the control and/or regulation device is programmed to control and/or regulate a blood treatment device that is connected to a blood filter;
controlling and/or regulating the blood treatment device for blood treatment of a patient with a first setting set using the first input device for at least one treatment parameter;
checking whether an input has occurred using the second input device;
evaluating the input entered using the second input device and/or specifying a second setting for the at least one treatment parameter based on the input entered using the second input device; and
controlling or regulating the blood treatment device for the blood treatment of the patient with the second setting and/or controlling or regulating the blood treatment device while adjusting or changing the first setting according to the input or the second setting.

39. The method according to claim 38, wherein the treatment parameter is chosen from the group consisting of: flow of dialysis liquid; blood flow through the blood filter; inflow of an infusion solution; concentration of osmotically active substances in the dialysis liquid; concentration of bicarbonate, sodium, potassium, magnesium and/or calcium in the dialysis liquid; filtration rate; ultrafiltration rate; time period of the blood treatment; and temperature of the dialysis liquid.

40. A system comprising:
a blood treatment device that comprises an extracorporeal blood circuit and a blood filter, or that is connected to an extracorporeal blood circuit and a blood filter, the blood treatment device further comprising or being connected to via signal link at least one control and/or regulation device that is programmed to control and/or regulate the blood treatment device by executing a method comprising:
controlling and/or regulating the blood treatment device for a blood treatment of a patient with a first setting set using a first input device for at least one treatment parameter;
checking whether an input has occurred using the second input device;
evaluating the input entered using the second input device and/or specifying a second setting for the at least one treatment parameter based on the input entered using the second input device; and
controlling or regulating the blood treatment device for the blood treatment of the patient with the second setting and/or controlling or regulating the blood treatment device while adjusting or changing the first setting according to the input or the second setting.

41. The system according to claim 40, comprising
a first input device, which is operable by a clinician for adjusting or changing settings of at least one treatment parameter, the first input device being connected via signal link to the at least one control and/or regulation device; and
a second input device, which is operable by a patient for adjusting or changing of settings of at least one treatment parameter, the second input device being connected via signal link to the at least one control and/or regulation device.

42. The system according to claim 40, wherein the blood treatment device is a hemodialysis device, a hemofiltration device, or a hemodiafiltration device.
